(19) United States
(12) Patent Application Publication
(10) Pub. No.: US 2021/0015375 A1
(43) Pub. Date: Jan. 21, 2021

(54) METHOD AND APPARATUS FOR MONITORING FETAL HEART RATE

(71) Applicants: UNIVERSITY OF ULSAN FOUNDATION FOR INDUSTRY COOPERATION, Ulsan (KR); THE ASAN FOUNDATION, Seoul (KR)

(72) Inventors: Chong Jai KIM, Seoul (KR); Eun Na KIM, Seoul (KR)

(73) Assignees: UNIVERSITY OF ULSAN FOUNDATION FOR INDUSTRY COOPERATION, Ulsan (KR); THE ASAN FOUNDATION, Seoul (KR)

(21) Appl. No.: 16/976,244

(22) PCT Filed: Mar. 27, 2019
(86) PCT No.: PCT/KR2019/003598

§ 371 Date: Aug. 27, 2020

(30) Foreign Application Priority Data
Mar. 29, 2018 (KR) 10-2018-0036762
Jun. 18, 2018 (KR) 10-2018-0069783

(51) Int. Cl. A61B 5/024; A61B 5/00

(52) U.S. Cl. CPC A61B 5/02411; A61B 5/7275; A61B 5/4362; A61B 5/7267

(57) ABSTRACT

A method for monitoring a fetal heart rate may include the steps of: acquiring fetal heart rate monitoring data; determining a fetal heart rate value by dividing the acquired fetal heart rate monitoring data by a predetermined time interval; and determining a fetal state by applying, to the determined fetal heartbeat value, a learned artificial intelligence algorithm using a learning database including the fetal heartbeat monitoring data pre-acquired in association with a plurality of fetuses.

### TECHNICAL FIELD

The present disclosure relates to a method for monitoring a fetal heart rate using an artificial intelligence algorithm trained with a learning database.

### BACKGROUND

Conventionally, in hospitals, in order to continuously detect the condition of a fetus for a predetermined time, the heart rate of the fetus is monitored using an electronic fetal heart rate monitoring test (hereinafter, referred to as a non-stress test (NST)). The NST is for detecting the fetal condition in a non-invasive way by attaching, to a mother's abdomen, a sensor for measuring the fetal heart rate. In the NST, a monitoring result sheet showing the heart rate status of the fetus is output, and a doctor or nurse analyzes it to assess the fetal condition. Meanwhile, the monitoring result sheet is output in a vast amount over time, which is why there is a practical limit for the doctor or nurse to accurately and thoroughly analyze the vast amount of monitoring result sheets.

In addition, when interpreting the NST, the doctor or nurse looks at the shape of the graph in the monitoring result sheet and assesses the fetal condition with a subjective interpretation based on his or her experience. However, assessing the fetal condition on the basis of the subjective interpretation may have problems that its accuracy is poor and errors may occur depending on the interpreter's condition or the like. Accordingly, a method for solving these problems is required.

### SUMMARY

A present disclosure provides a fetal heart rate monitoring technique capable of solving the limitations of the prior art as described above. More specifically, the fetal heart rate monitoring technique may generate a learning database using fetal heart rate information, and monitor the heart rate of a fetus of a high-risk mother using an artificial intelligence algorithm trained through the learning database, thereby more accurately detecting the condition of the fetus.

The present disclosure also provides an artificial intelligence fetal monitoring system that can precisely monitor a fetus of a high-risk mother to securely manage the high-risk mother, while overcoming shortages in maternity infrastructure and obstetricians and reducing damage to a newborn.

It is noted that aspects of the present disclosure are not limited to the above-mentioned aspects, and other unmentioned aspects of the present disclosure will be clearly understood by those skilled in the art from the following descriptions.

In accordance with one aspect of the present disclosure, there is provided a method for monitoring a fetal heart rate, comprising: acquiring fetal heart rate monitoring data, determining a fetal heart rate value by dividing the acquired fetal heart rate monitoring data at a predetermined time interval and determining a fetal condition by applying, to the determined fetal heart rate value, an artificial intelligence algorithm trained using a learning database including the fetal heart rate monitoring data previously acquired in association with a plurality of fetuses.

At least a portion of the plurality of fetuses may include fetuses having a miscarriage probability equal to or greater than a predetermined value, and a remaining portion of the plurality of fetuses includes fetuses having a miscarriage probability less than a predetermined value.

The learning database may include point data generated by dividing each of the previously acquired fetal heart rate monitoring data at the predetermined time interval, or representative point data generated by calculating an average based on a predetermined number of point data selected from the point data.

The determining of the fetal condition may comprise determining whether the miscarriage probability of the fetus is greater than or equal to a predetermined value based on the artificial intelligence algorithm applied to the fetal heart rate value.

The learning database may further include information on the fetal condition mapped for each of the point data, or information on the fetal condition mapped for each of the representative point data, and the determining of the fetal condition may comprise determining a fetal condition for each of fetal heart rate values divided at the predetermined time interval based on the artificial intelligence algorithm applied to the fetal heart rate value.

The method may further comprise outputting the determined fetal condition as an image divided into a plurality of blocks, wherein each of the plurality of blocks may represent a fetal condition determined for each of the fetal heart rate values.

Each of the plurality of blocks may be displayed in a color or pattern based on miscarriage probability sections to which the fetal condition belongs, and each of the miscarriage probability sections may previously be assigned a different color or pattern.

The method may further comprise: training the artificial intelligence algorithm by generating the learning database, wherein the training the artificial intelligence algorithm by generating the learning database may comprise: acquiring fetal heart rate monitoring data indicating a fetal heart rate for a certain time period for each of the plurality of fetuses, generating the point data by dividing the acquired fetal heart rate monitoring data at a predetermined time interval for each of the plurality of fetuses, determining whether a missing value is included in the point data, if the missing value is included, replacing the missing value with a point data value before or after the missing value and training the artificial intelligence algorithm using point data in which the missing value has been replaced.

The replacing of the missing value may comprise if the missing value is included, replacing the missing value by applying an artificial intelligence algorithm previously trained to supplement the missing value in the point data, and the artificial intelligence algorithm previously trained to supplement the missing value may be trained to infer the missing value based on pre-stored placental pathology images and fetal heart rate monitoring data.

In accordance with another aspect of the present disclosure, there is provided an apparatus for monitoring a fetal heart rate, comprising: a data acquisition unit configured to acquire fetal heart rate monitoring data, and determine a fetal heart rate value by dividing the acquired fetal heart rate monitoring data at a predetermined time interval and a data analysis unit configured to determine a fetal condition by applying, to the determined fetal heart rate value, an artificial intelligence algorithm trained using a learning database including the fetal heart rate monitoring data previously acquired in association with a plurality of fetuses.

At least a portion of the plurality of fetuses may include fetuses having a miscarriage probability equal to or greater than a predetermined value, and a remaining portion of the plurality of fetuses includes fetuses having a miscarriage probability less than a predetermined value.

The learning database may include point data generated by dividing each of the previously acquired fetal heart rate monitoring data at the predetermined time interval, or representative point data generated by calculating an average based on a predetermined number of point data selected from the point data.

The data analysis unit may determine whether the miscarriage probability of the fetus is greater than or equal to a predetermined value based on the artificial intelligence algorithm applied to the fetal heart rate value.

The learning database may further include information on the fetal condition mapped for each of the point data, or information on the fetal condition mapped for each of the representative point data, and the data analysis unit may determine a fetal condition for each of fetal heart rate values divided at the predetermined time interval based on the artificial intelligence algorithm applied to the fetal heart rate value.

The apparatus may further comprise: an output unit configured to output the determined fetal condition as an image divided into a plurality of blocks, wherein each of the plurality of blocks represents a fetal condition determined for each of the fetal heart rate values.

Each of the plurality of blocks may be displayed in a color or pattern based on miscarriage probability sections to which the fetal condition belongs, and each of the miscarriage probability sections may previously be assigned a different color or pattern.

The apparatus may further comprise: a learning unit configured to train the artificial intelligence algorithm by generating the learning database, wherein the learning unit acquires fetal heart rate monitoring data indicating a fetal heart rate for a certain time period for each of the plurality of fetuses, generates the point data by dividing the acquired fetal heart rate monitoring data at a predetermined time interval for each of the plurality of fetuses, determines whether a missing value is included in the point data, if the missing value is included, replaces the missing value with a point data value before or after the missing value, and trains the artificial intelligence algorithm using point data in which the missing value has been replaced.

The learning unit may replace, if the missing value is included, the missing value by applying an artificial intelligence algorithm previously trained to supplement the missing value in the point data, and the artificial intelligence algorithm previously trained to supplement the missing value may be trained to infer the missing value based on pre-stored placental pathology images and fetal heart rate monitoring data.

According to the present disclosure, it is possible to more accurately monitor the fetal heart rate using the artificial intelligence algorithm trained through the learning database related to the fetal heart rate.

According to the present disclosure, it is possible to more efficiently perform a fetal heart rate test by monitoring the fetal heart rate and providing the monitoring result as a single image in the form of a block to be grasped at a glance.

The effect of the present disclosure are not limited to the above-described effects and other effects which are not described herein will become apparent to those skilled in the art from the following description.

### BRIEF DESCRIPTION OF THE DRAWINGS

FIG. 1 is a view explaining the prior art of the present disclosure.

FIG. 2 is a view illustrating fetal heart rate monitoring data used to generate a learning database according to an embodiment of the present disclosure.

FIG. 3 shows an example of a functional configuration of a learning database generating device according to an embodiment of the present disclosure

FIG. 4 shows a flowchart of a method for generating the learning database according to an embodiment of the present disclosure

FIG. 5 shows an example of the fetal heart rate monitoring data for generating the learning database according to an embodiment of the present disclosure.

FIG. 6 shows an example of the learning database according to an embodiment of the present disclosure.

FIG. 7 shows an example of generating the learning database according to an embodiment of the present disclosure.

FIG. 8 shows another example of generating the learning database according to an embodiment of the present disclosure.

FIG. 9 shows an example of a functional configuration of a fetal heart rate monitoring device according to an embodiment of the present disclosure.

FIG. 10 shows a flowchart of the method for monitoring the fetal heart rate using the artificial intelligence according to an embodiment of the present disclosure.

FIG. 11 shows an example of an artificial intelligence algorithm according to an embodiment of the present disclosure.

FIG. 12 illustrates an example of a method for training the artificial intelligence algorithm according to an embodiment of the present disclosure.

FIG. 13 shows an example of an image output by the fetal heart rate monitoring device according to an embodiment of the present disclosure.

### DETAILED DESCRIPTION

The advantages and features of the present disclosure and the methods of accomplishing these will be clearly understood from the following description taken in conjunction with the accompanying drawings. However, embodiments are not limited to those embodiments described, as embodiments may be implemented in various forms. It should be noted that the present embodiments are provided to make a full disclosure and also to allow those skilled in the art to know the full range of the embodiments. Therefore, the embodiments are to be defined only by the scope of the appended claims.

In describing the embodiments of the present disclosure, if it is determined that detailed description of related known components or functions unnecessarily obscures the gist of the present disclosure, the detailed description thereof will be omitted. Further, the terminologies to be described below are defined in consideration of functions of the embodiments of the present disclosure and may vary depending on a user's or an operator's intention or practice. Accordingly, the definition thereof may be made on a basis of the content throughout the specification.

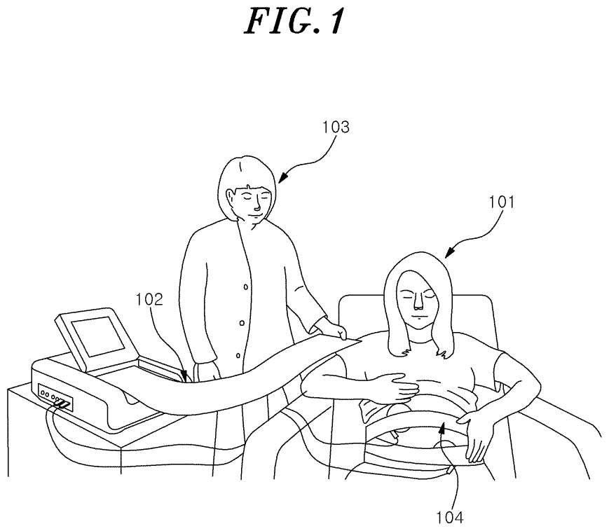

FIG. 1 is a view explaining the prior art of the present disclosure. More specifically, FIG. 1 is a view explaining a technique of determining the fetal condition using a non-stress test (NST).

Referring to FIG. 1, in the NST, a fetal heart rate may be measured by putting a belt 104 on the abdomen of a mother 101 to monitor the uterine contraction of the mother 101. Although not shown, the belt 104 may include a pressure transducer, and the fetal heart rate may be detected and measured by the pressure transducer. At this time, a fetal heart rate signal may be output onto a monitoring result sheet 102. Although not shown, information on the fetal heart rate signal may be represented in the form of a graph on the monitoring result sheet 102. According to an embodiment, the monitoring result sheet 102 may be displayed on an electronic device (e.g., a computer) in an image file format rather than a paper. A doctor 103 may determine the fetal condition by analyzing the monitoring result sheet 102. Meanwhile, the term “doctor 103” refers to a person (e.g., an expert in obstetrics) who can analyze the monitoring result sheet 102, but is not limited thereto.

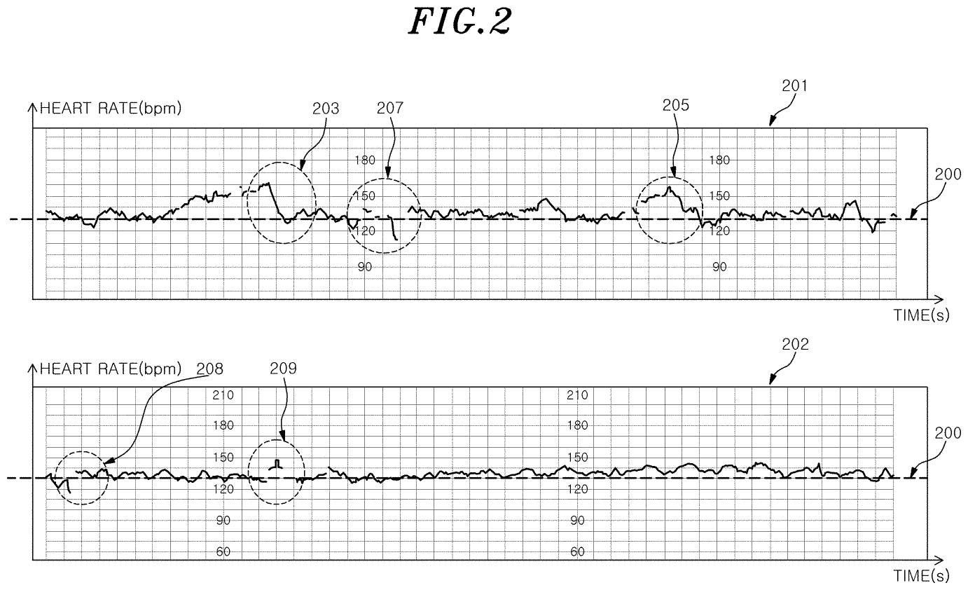

When the NST is performed in an existing hospital, the doctor 103 analyzes the monitoring result sheet 102 with a subjective interpretation based on his/her own experience to judge the fetal condition. Since there is no objective criterion for such subjective judgment, a problem may arise that the fetus may be at risk if the doctor's experience is insufficient. Embodiments of the present disclosure to be described below may provide a method and device capable of solving the above-mentioned problems. However, the problems that can be solved in the present disclosure are not limited to the above, and it is needless to say that various problems related to the fetal heart rate measurement can be solved. FIG. 2 is a view illustrating fetal heart rate monitoring data used to generate a learning database according to an embodiment of the present disclosure. FIG. 2 shows examples of monitoring data 201 when a fetus is in a normal (or reactive) condition and monitoring data 202 when a fetus is in an abnormal (or non-reactive) condition, in the NST.

In FIG. 2, the monitoring data 201 and 202 are shown in the form of an image, but according to an embodiment, the monitoring data 201 and 202 may be displayed in various forms such as numbers or codes.

In FIG. 2, the horizontal axis of the monitoring data 201 and 202 indicates time (s (seconds)), and the vertical axis thereof indicates heart rate (bpm (bit per minute)). In the monitoring data 201 and 202, the horizontal length of one rectangular cell may correspond to 10 seconds, and the vertical length thereof may correspond to 10 bpm. A baseline 200 may be determined as the median value between the maximum and the minimum of heart rate values measured for a predetermined time period (e.g., 10 minutes). For example, if the heart rate values measured for 10 minutes were between 120 bpm and 150 bpm, the baseline 200 may be determined as 135 bpm which is the median value between 120 bpm and 150 bpm.

When the fetus is in a normal condition, the heart rate value may vary by more than a predetermined value from the baseline 200 according to the fetal heart rate. For example, referring to the monitoring data 201, when the fetus is in a normal condition, a fetal heart rate graph may represent, as shown in part 203, the heart rate vertically increased by 1.5 cells, i.e., 15 bpm or more from the baseline 200. According to an embodiment, the fetal heart rate graph may represent the heart rate vertically decreased by 1.5 cells, i.e., 15 bpm or more from the baseline 200. When this change in the heart rate appears a predetermined number of times (e.g., 2 times) or more for a predetermined time period (e.g., 20 minutes), the fetal condition may be determined to be normal.

When the fetus is in an abnormal condition, the fetal heart may not be beating normally, and accordingly, the heart rate not changing over a certain range may continue for more than a certain time period. For example, referring to the monitoring data 202, when the fetus is in an abnormal condition, a state in which a vertical change in the heart rate is 1.5 cells or less may be maintained for a predetermined time period (e.g., 20 minutes) or more. That is, as shown in the monitoring data 202, a state in which a vertical change in the heart rate is 1.5 cells or less may continue in 20 or more horizontal cells.

According to an embodiment, the monitoring data 201, 202 may include missing values 207, 208, 209 due to failure in measurement in various situations such as when there is movement of the fetus in the uterus.

In an embodiment of the present disclosure to be described below, a learning database may be generated using the monitoring data 201 and 202, and an artificial intelligence algorithm may be trained using the generated learning database. In addition, the fetal condition may be more precisely interpreted by using the trained artificial intelligence algorithm.

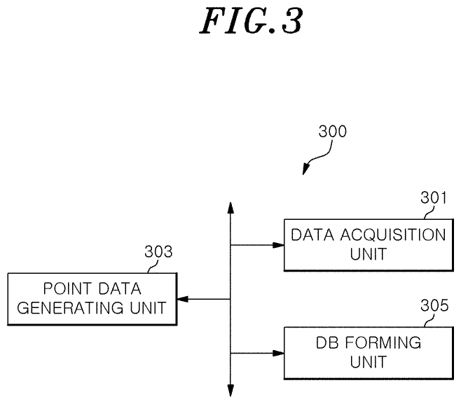

FIG. 3 shows an example of a functional configuration of a learning database generating device according to an embodiment of the present disclosure. The term ‘ . . . unit’ used below means a unit that processes at least one function or operation, which may be implemented by hardware, software, or a combination of hardware and software.

Referring to FIG. 3, a learning database generating device 300 may include a data acquisition unit 301, a point data generating unit 303, and a database (DB) forming unit 305. According to an embodiment, the data acquisition unit 301, the point data generating unit 303, and the DB forming unit 305 may each operate by an independent processor, or at least two or more of them may operate by one processor.

The data acquisition unit 301 may acquire (or collect) heart rate monitoring data for a plurality of fetuses by a user's input or by connection with other devices. Here, the plurality of fetuses may include fetuses in a normal condition (fetuses with a miscarriage probability less than a predetermined value) and fetuses in an abnormal condition (fetuses with a miscarriage probability greater than or equal to a predetermined value). The heart rate monitoring data of each fetus may include information on whether the fetus is in a normal condition or an abnormal condition.

Although not specifically shown, in some cases, placental pathology information may be added to the information on whether the fetus is in a normal condition or an abnormal condition. In this case, the reading of the heart rate monitoring data may be advanced.

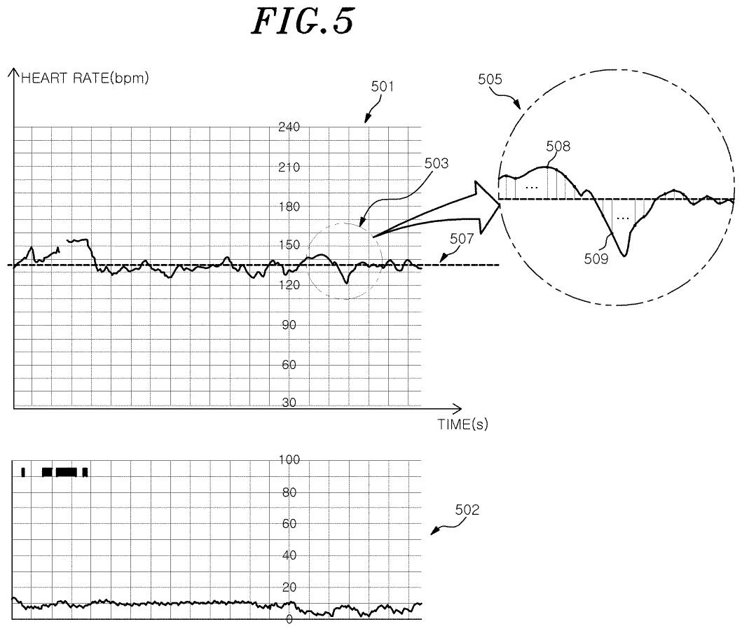

According to an embodiment, the data acquisition unit 301 may acquire the fetal heart rate monitoring data in which data obtained through a sensor (e.g., a pressure transducer) attached to a mother's abdomen is represented in an analog format. The analog format may be, for example, an image displayed in a graph form. In addition, according to an embodiment, when receiving the fetal heart rate monitoring data output in a paper form, the data acquisition unit 301 may scan the fetal heart rate monitoring data to acquire it in an image format. The acquisition of the fetal heart rate monitoring data by the data acquisition unit 301 is not limited to the above-described example and may be performed in various ways. See FIG. 5 for a more detailed description of the fetal heart rate monitoring data acquired by the data acquisition unit 301.

The point data generating unit 303 may generate point data by dividing each of a plurality of fetal heart rate monitoring data at a predetermined time interval. The point data generating unit 303 may sample the plurality of fetal heart rate monitoring data at the predetermined time interval to generate the point data indicating a fetal heart rate at the predetermined time interval. The predetermined time interval may be a predetermined value, e.g., 0.5 seconds. According to an embodiment, the point data generating unit 303 may calculate an average of the point data in each section including a predetermined number of point data, generate representative point data in the each section, and replace the point data with the representative point data.

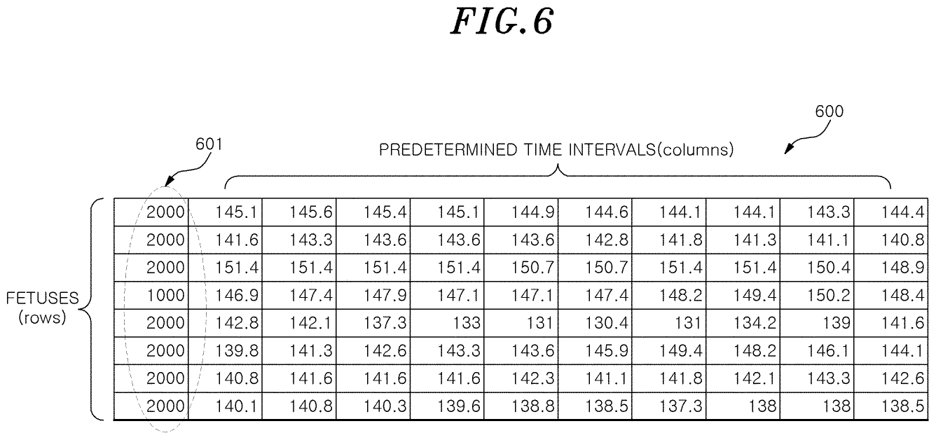

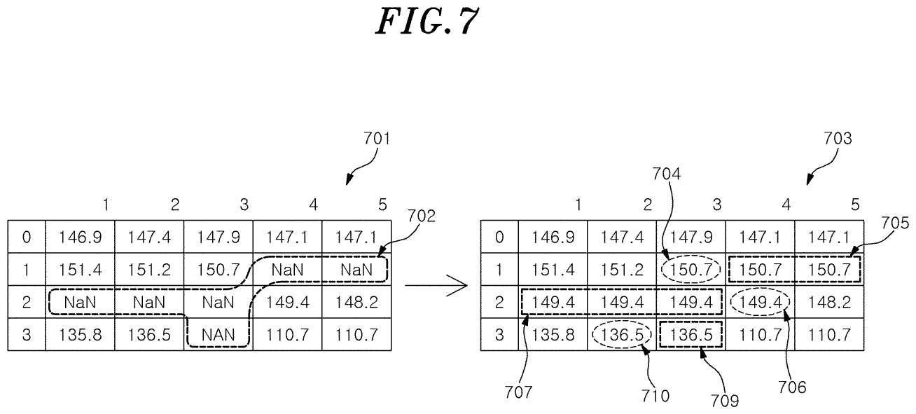

The DB forming unit 305 may form a learning database using the point data. In some cases, when a missing value (e.g., a missing value 207, 208, 209) is included at a particular time point of the fetal heart rate monitoring data, a missing value may also be included in the point data. In this case, the DB forming unit 305 may make up for the missing value to form the learning database. For example, if point data exists at a time point before a missing value, the DB forming unit 305 may replace the missing value with the point data at the time point before the missing value. For another example, if point data exists at a time point after a missing value, the DB forming unit 305 may replace the missing value with the point data at the time point after the missing value. See FIG. 6 for a more detailed description of the learning database and see FIG. 7 for a more detailed description related to the replacement of the missing value.

In some cases, the DB forming unit 305 may infer a missing value using an artificial intelligence algorithm. Specifically, the DB forming unit 305 may include the artificial intelligence algorithm trained using the placental pathology information and previously-acquired fetal heart rate monitoring data to infer the missing value. Such an artificial intelligence algorithm may be trained, if there is a missing value in the fetal heart rate monitoring data, to infer the missing value using the fetal heart rate monitoring data and placental pathology images. In this case, the DB forming unit 305 may estimate the missing value by applying the artificial intelligence algorithm to the fetal heart rate monitoring data having the missing value.

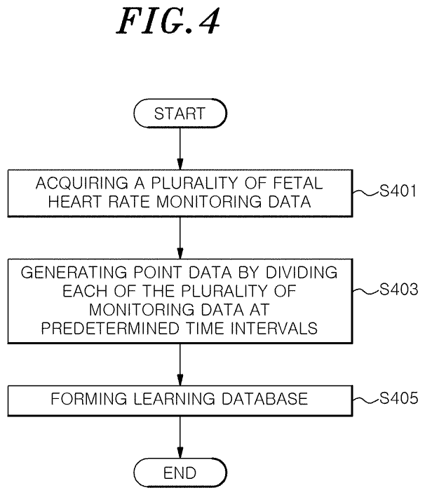

FIG. 4 shows a flowchart of a method for generating the learning database according to an embodiment of the present disclosure.

Referring to FIG. 4, the data acquisition unit 301 may acquire (or collect) a plurality of fetal heart rate monitoring data (hereinafter, a plurality of monitoring data) (step S401). The plurality of monitoring data may include heart rate monitoring data of two or more fetuses. The plurality of monitoring data may include information on the condition of fetuses, and data obtained by monitoring the heart rates of the fetuses for a certain period of time.

The data acquisition unit 301 may acquire the plurality of monitoring data in various ways. For example, the data acquisition unit 301 may acquire the plurality of monitoring data by a user's input, or from another device (e.g., an NST device or an external device having NST results) based on the connection with the another device. For another example, when receiving the fetal heart rate monitoring data output in a paper form, the data acquisition unit 301 may scan the received fetal heart rate monitoring data to acquire it in an analog format (e.g., an image).

The point data generating unit 303 may generate point data by dividing each of the plurality of monitoring data at a predetermined time interval (step S403). The point data generating unit 303 may divide the acquired plurality of monitoring data at predetermined time interval to generate the point data corresponding to each of the divided intervals. According to an embodiment, the acquired plurality of monitoring data may be analog data (e.g., an image). In this case, the point data generating unit 303 may identify and divide the analog data at a predetermined time interval, and then may derive a point data value matched for each of the divided time intervals from the analog data. The point data may be a fetal heart rate measurement value representing each of the divided time intervals.

According to an embodiment, the point data generating unit 303 may calculate an average of the point data in each section including a predetermined number of point data, and generate representative point data in the each section. For example, the point data generating unit 303 may generate 20 representative point data by bundling 100 point data generated at 0.1 second intervals by 5 point data. In this case, ultimately, the point data generating unit 303 may generate the representative point data at 0.5 second intervals and replace the point data with the representative point data (or use the representative point data as the point data). See FIG. 5 for a more detailed description related to the generation of the point data.

The DB forming unit 305 may form the learning database. The DB forming unit 305 may form the learning database using the point data. The DB forming unit 305 may classify the point data for each of a plurality of fetuses to form the learning database. For example, the DB forming unit 305 may divide the plurality of fetuses into rows, and divide the point data into columns in temporal order to form the learning database. See FIG. 6 for a more detailed description of the learning database.

When the point data includes a missing value, the DB forming unit 305 may make up for the missing value to form the learning database. For example, when a plurality of point data continuously includes a missing value, the DB forming unit 305 may replace the missing value with a point data value before or after the missing value. See FIG. 7 for a more detailed description related to the replacement of the missing value.

FIG. 5 shows an example of the fetal heart rate monitoring data for generating the learning database according to an embodiment of the present disclosure. FIG. 5 may be an example of the monitoring data derived by the NST. After the NST, monitoring data 501 indicating the heart rate of a fetus and monitoring data 502 indicating the uterine contraction of a mother over time may be derived.

Referring to FIG. 5, since the change of uterine contraction is insignificant in the monitoring data 502, it can be seen that the monitoring data 501 is the fetal heart rate monitoring data when the uterus is not contracted. Therefore, hereinafter, the acquisition of the fetal heart rate monitoring data during the non-contraction of the uterus will be described, but the present disclosure is not limited thereto, and the fetal heart rate monitoring data may be acquired in a similar manner even during the contraction of the uterus.

In FIG. 5, the generation of the point data by the point data generating unit 303 will be described using part 503 as an example. The point data generating unit 303 may divide part 503 at a predetermined time interval to generate, as the point data, a heart rate value corresponding to each of the time intervals. The predetermined time interval may be a predetermined value, e.g., 0.5 seconds, but is not limited to the example described herein.

Referring to FIG. 5, part 505 is an enlarged view of part 503, and the point data may be heart rate values corresponding to respective points located on the graph in part 505.

The point data generating unit 303 according to an embodiment of the present disclosure may set an actual heart rate value as a point data value, or set the difference between a baseline 507 and a heart rate value based on the value of the baseline 507 as a point data value. For example, the point data generating unit 303 may generate point data 508 having a value of 137 bpm and point data 509 having a value of 121 bpm. In this case, according to an embodiment, the DB forming unit 305 may form the learning database by mapping the value of the baseline 507 to the generated point data. For another example, the point data generating unit 303 may, based on the value (135 bpm) of the baseline 507, generate point data 508 having a value of 2 bpm and point data 509 having a value of −13 bpm.

The point data generating unit 303 according to an embodiment of the present disclosure may, based on the acquired heart rate monitoring data of the plurality of fetuses, generate a plurality of point data at a predetermined time interval for each fetus. For example, when acquiring the heart rate monitoring data for two fetuses, the point data generating unit 303 may generate the point data for each fetus. At this time, the point data generating unit 303 may generate the point data for the acquired heart rate monitoring data without distinguishing whether the fetus is in a normal condition or an abnormal condition. However, since a value for the fetal condition may be included in the heart rate monitoring data, the DB forming unit 305 may map this information into each point data to generate the learning database. See FIG. 6 for a detailed description of the learning database.

When the heart rate monitoring data is in a format unable to be analyzed, the point data generating unit 303 according to an embodiment of the present disclosure may convert the heart rate monitoring data into an analyzable format in order to generate the point data. For example, if the monitoring data is in a format (e.g., a portable document format (PDF)) unable to be analyzed, it may be converted into an image format (e.g., a graphic interchange format (gif)). The point data generating unit 303 may generate the point data by dividing a graph displayed in an image format file at predetermined time intervals (e.g., 0.5 seconds) or predetermined pixel intervals (e.g., 3 pixels). If the heart rate value changes significantly at a time point corresponding to the predetermined time interval, the point data may be extracted as an optimal value by a pre-designated method. The pre-designated method may be, for example, a method of, when the heart rate value increases from 100 bpm to 140 bpm at 0.5 seconds, determining the corresponding point data as an intermediate value between 100 bpm and 140 bpm. For another example, the pre-designated method may include a method of determining, as the point data, the value of one of the top 25% (i.e., 110 bpm) or the bottom 75% (i.e., 130 bpm) between 100 bpm and 140 bpm.

FIG. 6 shows an example of the learning database according to an embodiment of the present disclosure.

Referring to FIG. 6, a learning database 600 may be generated in a form that each column corresponds to a predetermined time interval, and each row corresponds to each fetus. For example, first to third rows may indicate the point data of first to third fetuses, respectively. In addition, a first column may be a first time point (e.g., 0.5 seconds) corresponding to an initial one of predetermined time intervals (e.g., 0.5 seconds), a second column may be a second time point (e.g., 1 second) that follows the first time point, and a third column may be a third time point (e.g., 1.5 seconds) that follows the second time point.

The learning database 600 may include data representing the condition of each fetus. More specifically, the learning database 600 may include condition information 601 of each fetus. In the condition information 601, reference numeral 1000 may indicate a case where a fetus is in a normal condition, and reference numeral 2000 may indicate a case where a fetus is in an abnormal condition. The condition information 601 may be represented in various forms (such as other numbers or letters) that can represent the condition of each fetus, and is not limited to the illustrated example.

The learning database 600 illustrated in the drawing is generated based on the point data determined as the actually measured fetal heart rate values. However, according to an embodiment, as described above with reference to FIG. 5, the point data may be generated based on a difference from the baseline 507, and in this case, the learning database 600 may be generated in the form of positive and negative values from the baseline 507.

FIG. 7 shows an example of generating the learning database according to an embodiment of the present disclosure. FIG. 7 is a diagram explaining an example of the learning database generated when a missing value is included in the point data.

Referring to FIG. 7, a learning database 701 may include missing values 702. In this case, the DB forming unit 305 may make up for the missing values of the learning database 701. For example, the DB forming unit 305 may replace the missing value with a value nearest to the missing value among data located in the same row. That is, the DB forming unit 305 may replace the missing value with the same value as a value before or after the missing value among data located in the same row.

The DB forming unit 305 according to an embodiment of the present disclosure may basically replace the missing value with the point data located before the missing value, but when there is no point data before the missing value, with the point data after the missing value. In FIG. 7, referring to a learning database 703 in which the missing values have been replaced, the DB forming unit 305 may fill missing values 705 with point data 704 located before the missing values 705. Since there is no point data before missing values 707, the DB forming unit 305 may fill the missing values 707 with point data 706 located after the missing values 707. In this way, the DB forming unit 305 may fill a missing value 709 with point data 710 located before the missing value 709.

Meanwhile, a method of replacing a missing value may exist in various ways, and is not limited to the example described with reference to FIG. 7. For example, the missing value is basically replaced with the point data located after the missing value, but when there is no point data after the missing value, the missing value may be replaced with the point data before the missing value. For another example, when a plurality of consecutive missing values exist, the missing values may be sequentially replaced with the point data adjacent to each missing value among the point data located in the same row. That is, if there are four consecutive missing values and there are point data before and after the four consecutive missing values, the former two of the consecutive missing values may be replaced with the point data before them, and the latter two of the consecutive missing values may be replaced with the point data after them.

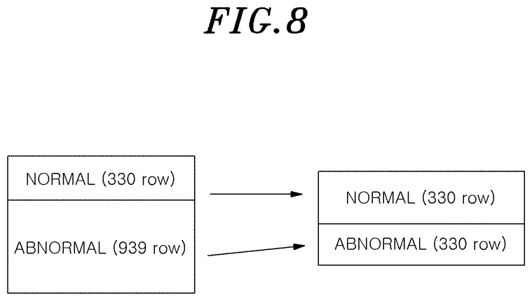

FIG. 8 shows another example of generating the learning database according to an embodiment of the present disclosure. Based on the heart rate monitoring data acquired by the data acquisition unit 301, when the proportions of a normal condition and an abnormal condition for a plurality of fetuses are different, the proportions are adjusted to generate a more sophisticated learning database, which is exemplarily illustrated in FIG. 8.

Referring to FIG. 8, when the acquired heart rate monitoring data is for 330 fetuses of a normal condition and 939 fetuses of an abnormal condition, data having more samples, i.e., the data for 939 fetuses of an abnormal condition may be downsampled to a size of 330 fetuses. Here, downsampling may refer to an operation of adjusting the number of data in order to equalize the proportions of the fetuses of a normal condition and an abnormal condition.

Downsampling according to an embodiment of the present disclosure may be performed in various forms. For example, the downsampling may be performed by first removing the fetal data having many missing values. For another example, the downsampling may be performed by randomly selecting data of 330 fetuses and removing the remaining data.

The downsampling according to an embodiment of the present disclosure may be performed in association with any one of the steps for generating the learning database. For example, the downsampling may be performed immediately after data is acquired by the data acquisition unit 301. For another example, the downsampling may be performed after the point data is generated by the point data generating unit 303. For another example, the downsampling may be performed according to the presence or absence (or the number) of missing values that has been determined by the DB forming unit 305.

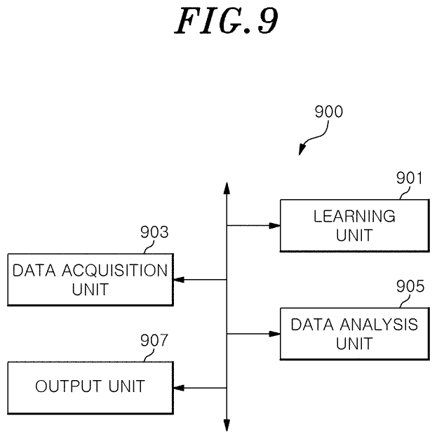

FIG. 9 shows an example of a functional configuration of a fetal heart rate monitoring device according to an embodiment of the present disclosure. FIG. 9 includes an example of a functional configuration of a fetal heart rate monitoring device (hereinafter, a monitoring device) 900 using the artificial intelligence algorithm. The term ‘ . . . unit’ used below means a unit that processes at least one function or operation, which may be implemented by hardware, software, or a combination of hardware and software.

Referring to FIG. 9, a monitoring device 900 may include a learning unit 901, a data acquisition unit 903, a data analysis unit 905, and an output unit 907. Although not illustrated, according to an embodiment, the monitoring device 900 may include the learning database generating device 300 of FIG. 3 as a component.

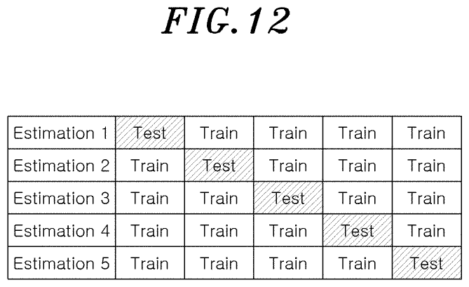

The learning unit 901 may train the artificial intelligence algorithm using the learning database generated by the learning database generating device 300. The learning unit 901 may train the artificial intelligence algorithm to more accurately determine the fetal condition using the learning database generated by the learning database generating device 300. According to an embodiment, when a missing value is included in the learning database, the learning unit 901 may supplement the missing value of the learning database. In this case, the learning unit 901 may train the artificial intelligence algorithm using the supplemented learning database. See FIG. 12 for a more detailed description related to the training of the artificial intelligence algorithm.

The data acquisition unit 903 may acquire the fetal heart rate monitoring data (e.g., monitoring data 201 and 202). The data acquisition unit 903 may acquire the fetal heart rate monitoring data in real time from a sensor (e.g., a pressure transducer) for detecting a fetal heart rate, the sensor being attached to a mother's abdomen.

The data analysis unit 905 may determine the fetal condition by identifying the fetal heart rate monitoring data acquired in real time, using the trained artificial intelligence algorithm.

The output unit 907 may output the fetal condition as an image divided into a plurality of blocks. Each of the plurality of blocks may indicate a fetal condition determined for each of fetal heart rate values. Specifically, each of the plurality of blocks may be displayed in a color or pattern based on miscarriage probability sections to which the fetal condition belongs.

At this time, each of the miscarriage probability sections may be previously assigned a different color or pattern. For example, when the fetal condition is displayed in color, red may indicate a stable fetal condition section in which the probability of miscarriage is lower than a predetermined value, and blue may indicate a dangerous fetal condition section in which the probability of miscarriage is higher than a predetermined value.

The monitoring device 900 according to an embodiment of the present disclosure may determine the fetal condition in real time by more accurately analyzing the fetal heart rate monitoring data based on the artificial intelligence algorithm trained based on the learning database.

The monitoring device 900 according to an embodiment of the present disclosure may provide an analysis with accuracy equal to or higher than the professional level of obstetricians, when the fetal heart rate needs to be monitored in a situation where there is no obstetrician during the night hours or an area where there is no obstetric hospital, or when a midwife or nurse needs to analyze the fetal heart rate monitoring data. In addition, in the event of a fetal emergency during the monitoring of the fetal heart rate in hospitals with poor maternity facilities, it is possible to activate an emergency delivery system by identifying the nearest hospital capable of emergency delivery of a high-risk mother and transmitting data to the corresponding hospital.

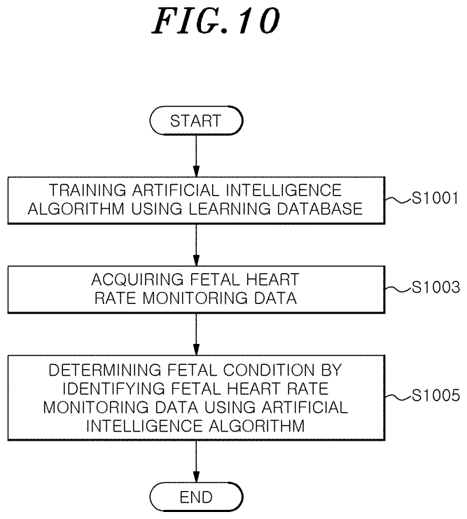

FIG. 10 shows a flowchart of the method for monitoring the fetal heart rate using the artificial intelligence according to an embodiment of the present disclosure.

The learning unit 901 may train the artificial intelligence algorithm using the learning database (step S1001). The learning unit 901 may train the artificial intelligence algorithm to more accurately determine the fetal condition using the learning database generated by the learning database generating device 300. For example, the learning unit 901 may train the artificial intelligence algorithm to determine the fetal condition as a normal condition when a variation in fetal heart rate during a predetermined time period (e.g., 20 minutes) continuously exceeds a certain value (e.g., 15 bpm) for a certain time period (e.g., 15 seconds). For another example, the learning unit 901 may train the artificial intelligence algorithm to determine the fetal condition as an abnormal condition when there is no section in which a variation in fetal heart rate during a predetermined time period (e.g., 20 minutes) continuously exceeds a certain value (e.g., 15 bpm) for a certain time period (e.g., 15 seconds). See FIG. 12 for a more detailed description related to the training of the artificial intelligence algorithm.

The data acquisition unit 903 may acquire the fetal heart rate monitoring data (step S1003). The data acquisition unit 301 may acquire the fetal heart rate monitoring data in real time from a sensor for detecting a fetal heart rate, the sensor being attached to a mother's abdomen. The fetal heart rate monitoring data may be analog data or digital data. For example, the digital data may be the fetal heart rate values measured by the sensor. For another example, the analog data may, when an image in a paper form is scanned, be a scanned picture. In this case, the data acquisition unit 903 may identify the scanned picture to acquire the fetal heart rate values.

The data acquisition unit 903 may determine the fetal heart rate values at predetermined time intervals (0.5 seconds). For another example, a moving average may be calculated for each predetermined number (e.g., five) of the fetal heart rate values at predetermined time intervals to determine each moving average as the fetal heart rate value.

The data analysis unit 905 may identify the fetal heart rate monitoring data to determine the fetal condition using the artificial intelligence algorithm (step S1005). The data analysis unit 905 may determine whether the fetal condition is normal or abnormal by identifying the fetal heart rate monitoring data acquired in real time using the artificial intelligence algorithm.

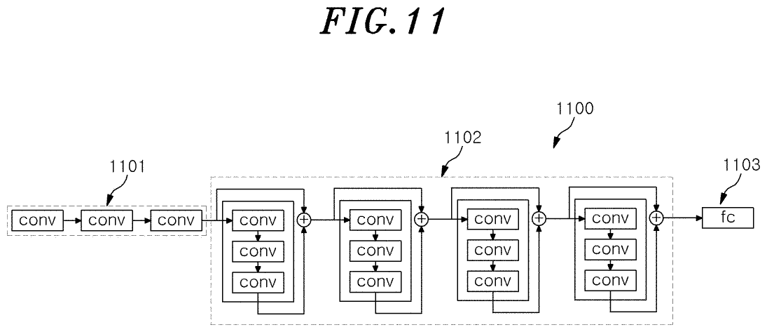

FIG. 11 shows an example of an artificial intelligence algorithm according to an embodiment of the present disclosure. Referring to FIG. 11, an artificial intelligence algorithm 1100 may be one-dimensional convolution neural network (1D-CNN, or 1D ResNet). A detailed description of parts related to the prior art in each component of the artificial intelligence algorithm may be omitted.

The artificial intelligence algorithm 1100 according to an embodiment of the present disclosure may include three consecutive convolutional layers 1101, four ResNet blocks 1102, and one fully connected layer 1103. Each of the ResNet blocks may include three convolutional layers, and a skip connection that directly connects the input of the ResNet block to the output thereof may be used to perform deeper learning. The fully connected layer 1103 may output its input value as a result of Group 1 (e.g., fetuses in a normal condition) or Group 2 (e.g., fetuses in an abnormal condition).

FIG. 12 illustrates an example of a method for training the artificial intelligence algorithm according to an embodiment of the present disclosure. Referring to FIG. 12, the artificial intelligence algorithm may be trained based on the learning database divided into five groups.

As illustrated in FIG. 12, the learning database may be divided into four learning data groups for training the artificial intelligence algorithm and one verification data group for determining whether the training is successful. The training may be performed five times, and in this case, the one group used as verification data may be changed each turn.

The learning unit 901 according to an embodiment of the present disclosure may train the artificial intelligence algorithm five times using the learning database divided into five groups. The verification data groups used in each of the five training processes may be different from each other. The monitoring device 900 may analyze the fetal heart rate data more accurately by using the trained artificial intelligence algorithm.

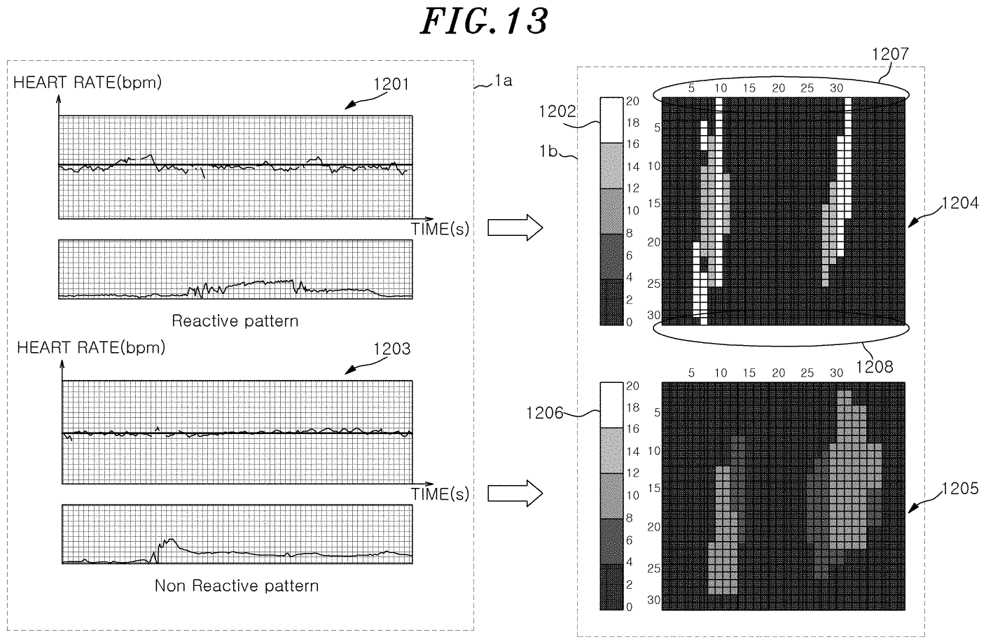

FIG. 13 shows an example of an image output by the fetal heart rate monitoring device according to an embodiment of the present disclosure. Specifically, in the example of FIG. 13, information on the fetal condition obtained by analyzing fetal heart rate values is provided in the form of a single image.

Reference numeral 1a represents an example of the fetal heart rate monitoring data described with reference to FIG. 5. The fetal heart rate monitoring data such as reference numeral 1a may be obtained based on what has been described with reference to FIGS. 1 to 12.

In the reference numeral 1a, a graph 1201 may be the heart rate monitoring data for a fetus of a reactive condition, and a graph 1203 may be the heart rate monitoring data for a fetus of a non-reactive condition.

The data acquisition unit 903 may divide the fetal heart rate monitoring data at predetermined a time interval to obtain the fetal heart rate values. When the fetal heart rate values are input to the artificial intelligence algorithm, the fetal condition (or risk level) may be determined for each of the fetal heart rate values. The artificial intelligence algorithm used at this time may be a pre-trained algorithm to determine the fetal condition.

The fetal heart rate values include a plurality of heart rate values divided at a predetermined time interval, and the plurality of heart rate values may be divided into a plurality of blocks and displayed on the image. Each of the plurality of blocks may correspond to the fetal condition of each predetermined time period. Accordingly, a finally derived image may be the same as reference numeral 1b.

Meanwhile, the fetal condition may be different for each of the fetal heart rate values. Based on this, as shown in the reference numeral 1b, the fetal condition may be output in the form of a single image to facilitate the representation of the fetal condition for each fetal heart rate value. For example, as shown in the drawing, the plurality of blocks, each of which is a section indicating each fetal heart rate value, may each be arranged according to time and may be output as a single image.

The reference numeral 1b may be an image derived from the reference numeral 1a. Specifically, an image 1204 may be derived using raw data extracted from the graph 1201, and an image 1205 may be derived using raw data extracted from the graph 1203.

The raw data extracted from each of the graphs 1201 and 1203 may be 1 to 960 signal values. Based on these values, the image may be composed of a 30×32 matrix. Each block constituting the matrix may be displayed on a scale of 0 to 20 as indicated by rectangular bars 1202 and 1206 illustrated adjacent to the matrix.

Here, the scale is a value indicating the fetal heart rate value (or the fetal condition). For visual representation, the color, contrast, and pattern of the block may be pre-specified according to the scale value, and these pre-specified contents may be displayed through the rectangular bars 1202 and 1206. Based on the rectangular bar 1202, a user provided with the image of the reference numeral 1b may easily grasp the fetal condition at a glance.

Further, in the reference numeral 1b, the scale is represented by different contrasts depending on its value, but is not limited thereto, and may be displayed in different colors or different patterns depending on the value. Furthermore, the image is not limited to the example of the reference numeral 1b, and of course, the image may be represented on various scales for matrices of various sizes.

Each of the plurality of blocks constituting the image may be displayed in various ways. For example, it may be arranged in various ways or represented in various forms. Specifically, for example, each of the plurality of blocks may be displayed in different colors depending on the fetal condition. For another example, the plurality of blocks may be displayed in different colors depending on a section (e.g., miscarriage probability section) to which the fetal condition belongs. That is, when one block is displayed in red, it may mean that the probability of miscarriage corresponds to a first section in the graph of the corresponding one minute. However, the method of representing the fetal condition is not limited to color, and the fetal condition may be represented by various patterns, or the shapes, sizes, and types of the blocks.

The plurality of blocks may be arranged in various ways. For example, as shown in the reference numeral 1b, when a first portion 1207 is the top portion of the image and a second portion 1208 is the bottom portion of the image, the plurality of blocks may be arranged in temporal order from the top of the image to the bottom thereof, and then from the left of the image to the right thereof. That is, the plurality of blocks may be divided into sections of 30 blocks to be arranged in temporal order from the top to the bottom in the image. Accordingly, a first block of each section, such as a 1^{st} block, a 31^{st} block and a 61^{st} block, may be displayed in the first row.

Such arrangement method or display form of the blocks may be specified in advance, and by providing information on the fetal condition in one image, the fetal condition may be grasped at a glance.

Since the present disclosure may be applied to all pregnant women, the range of the market may be very wide when commercialized. That is, the fetal heart rate monitoring analysis system according to the present disclosure may be supplied worldwide.

The present disclosure can provide an analysis with accuracy equal to or higher than the professional level of obstetricians, when the fetal heart rate needs to be monitored in a situation where there is no obstetrician during the night hours or an area where there is no obstetric hospital, or when a midwife or nurse needs to analyze the fetal heart rate monitoring data. In addition, in the event of a fetal emergency during the monitoring of the fetal heart rate in hospitals with poor maternity facilities, it is possible to activate an emergency delivery system by transmitting data to the nearest hospital capable of emergency delivery of a high-risk mother.

The present disclosure can enable doctors in night time, rural areas, or island areas to safely keep the pregnancy of pregnant women having complications. According to the present disclosure, doctors may reduce fetal damage by providing rapid first aid in situations where a mother or a fetus is at risk during delivery. In addition, women's anxiety about pregnancy and delivery may be reduced.

The combinations of the respective blocks of a block diagram and the combinations of the respective sequences of a flow diagram attached herein may be carried out by computer program instructions. Since the computer program instructions may be executed by the processor of a general purpose computer, a special purpose computer, or other programmable data processing apparatus, the instructions, executed by the processor of the computer or other programmable data processing apparatus, create means for performing functions described in the respective sequences of the flow diagram or the respective blocks of the block diagram. The computer program instructions, in order to implement functions in a specific manner, may be stored in a computer-readable storage medium or a computer-useable storage medium for other programmable data processing apparatus, and the instructions stored in the computer-readable storage medium or the computer-useable storage medium may produce manufacturing items that include means for instructions to perform the functions described in the respective sequences of the flow diagram or the respective blocks of the block diagram. The computer program instructions may be loaded in a computer or other programmable data processing apparatus, and therefore, the instructions, which are a series of sequences executed in a computer or other programmable data processing apparatus to create processes executed by a computer to operate a computer or other programmable data processing apparatus, may provide operations for executing functions described in the respective sequences of the flow diagram or the respective blocks of the block diagram.

Moreover, the respective block or the respective sequences may refer to two or more modules, segments, or codes including at least one executable instruction for executing a specific logic function(s). In some alternative embodiments, it is noted that the functions described in the sequences may be run out of order. For example, two consecutive sequences may be executed simultaneously or in reverse order according to the particular function.

The above description illustrates the technical idea of the present invention, and it will be understood by those skilled in the art to which this present invention belongs that various changes and modifications may be made without departing from the scope of the essential characteristics of the present invention. Therefore, the exemplary embodiments disclosed herein are not used to limit the technical idea of the present invention, but to explain the present invention, and the scope of the technical idea of the present invention is not limited by those embodiments. Therefore, the scope of protection of the present invention should be construed as defined in the following claims, and all technical ideas that fall within the technical idea of the present invention are intended to be embraced by the scope of the claims of the present invention.

1-18. (canceled)

19. A method for monitoring a fetal heart rate, comprising:
acquiring fetal heart rate monitoring data;
determining a fetal heart rate value by dividing the acquired fetal heart rate monitoring data at a predetermined time interval; and
determining a fetal condition by applying, to the determined fetal heart rate value, an artificial intelligence algorithm trained using a learning database including the fetal heart rate monitoring data previously acquired in association with a plurality of fetuses.

20. The method of claim 19, wherein at least a portion of the plurality of fetuses includes fetuses having a miscarriage probability equal to or greater than a predetermined value, and a remaining portion of the plurality of fetuses includes fetuses having a miscarriage probability less than a predetermined value.

21. The method of claim 20, wherein the learning database includes point data generated by dividing each of the previously acquired fetal heart rate monitoring data at the predetermined time interval, or representative point data generated by calculating an average based on a predetermined number of point data selected from the point data.

22. The method of claim 21, wherein the determining of the fetal condition comprises determining whether the miscarriage probability of the fetus is greater than or equal to a predetermined value based on the artificial intelligence algorithm applied to the fetal heart rate value.

23. The method of claim 22, wherein the learning database further includes information on the fetal condition mapped for each of the point data, or information on the fetal condition mapped for each of the representative point data, and
the determining of the fetal condition comprises determining a fetal condition for each of fetal heart rate values divided at the predetermined time interval based on the artificial intelligence algorithm applied to the fetal heart rate value.

24. The method of claim 23, further comprising:
outputting the determined fetal condition as an image divided into a plurality of blocks,
wherein each of the plurality of blocks represents a fetal condition determined for each of the fetal heart rate values.

25. The method of claim 24, wherein each of the plurality of blocks is displayed in a color or pattern based on miscarriage probability sections to which the fetal condition belongs, and
each of the miscarriage probability sections is previously assigned a different color or pattern.

26. The method of claim 19, further comprising:
training the artificial intelligence algorithm by generating the learning database,
wherein the training the artificial intelligence algorithm by generating the learning database comprises:
acquiring fetal heart rate monitoring data indicating a fetal heart rate for a certain time period for each of the plurality of fetuses;
generating the point data by dividing the acquired fetal heart rate monitoring data at a predetermined time interval for each of the plurality of fetuses;
determining whether a missing value is included in the point data;
if the missing value is included, replacing the missing value with a point data value before or after the missing value; and
training the artificial intelligence algorithm using point data in which the missing value has been replaced.

27. The method of claim 26, wherein the replacing of the missing value comprises, if the missing value is included, replacing the missing value by applying an artificial intelligence algorithm previously trained to supplement the missing value in the point data, and
the artificial intelligence algorithm previously trained to supplement the missing value is trained to infer the missing value based on pre-stored placental pathology images and fetal heart rate monitoring data.

28. An apparatus for monitoring a fetal heart rate, comprising:
a data acquisition unit configured to acquire fetal heart rate monitoring data, and determine a fetal heart rate value by dividing the acquired fetal heart rate monitoring data at a predetermined time interval; and
a data analysis unit configured to determine a fetal condition by applying, to the determined fetal heart rate value, an artificial intelligence algorithm trained using a learning database including the fetal heart rate monitoring data previously acquired in association with a plurality of fetuses.

29. The apparatus of claim 28, wherein at least a portion of the plurality of fetuses includes fetuses having a miscarriage probability equal to or greater than a predetermined value, and a remaining portion of the plurality of fetuses includes fetuses having a miscarriage probability less than a predetermined value.

30. The apparatus of claim 29, wherein the learning database includes point data generated by dividing each of the previously acquired fetal heart rate monitoring data at the predetermined time interval, or representative point data generated by calculating an average based on a predetermined number of point data selected from the point data.

31. The apparatus of claim 30, wherein the data analysis unit determines whether the miscarriage probability of the fetus is greater than or equal to a predetermined value based on the artificial intelligence algorithm applied to the fetal heart rate value.

32. The apparatus of claim 31, wherein the learning database further includes information on the fetal condition mapped for each of the point data, or information on the fetal condition mapped for each of the representative point data, and
the data analysis unit determines a fetal condition for each of fetal heart rate values divided at the predetermined time interval based on the artificial intelligence algorithm applied to the fetal heart rate value.

33. The apparatus of claim 32, further comprising:
an output unit configured to output the determined fetal condition as an image divided into a plurality of blocks,
wherein each of the plurality of blocks represents a fetal condition determined for each of the fetal heart rate values.

34. The apparatus of claim 33, wherein each of the plurality of blocks is displayed in a color or pattern based on miscarriage probability sections to which the fetal condition belongs, and
each of the miscarriage probability sections is previously assigned a different color or pattern.

35. The apparatus of claim 28, further comprising:
a learning unit configured to train the artificial intelligence algorithm by generating the learning database,
wherein the learning unit
acquires fetal heart rate monitoring data indicating a fetal heart rate for a certain time period for each of the plurality of fetuses,
generates the point data by dividing the acquired fetal heart rate monitoring data at a predetermined time interval for each of the plurality of fetuses,
determines whether a missing value is included in the point data,
if the missing value is included, replaces the missing value with a point data value before or after the missing value, and
trains the artificial intelligence algorithm using point data in which the missing value has been replaced.

36. The apparatus of claim 35, wherein the learning unit replaces, if the missing value is included, the missing value by applying an artificial intelligence algorithm previously trained to supplement the missing value in the point data, and
the artificial intelligence algorithm previously trained to supplement the missing value is trained to infer the missing value based on pre-stored placental pathology images and fetal heart rate monitoring data.
